(19) United States
(12) Patent Application Publication
(10) Pub. No.: US 2017/0135924 A1
(43) Pub. Date: May 18, 2017

(54) NON-PROPELLANT STATIC ELIMINATING SPRAY

(71) Applicant: Donna M. Baum, Lake in the Hills, IL (US)

(72) Inventor: Donna M. Baum, Lake in the Hills, IL (US)

(21) Appl. No.: 15/351,127

(22) Filed: Nov. 14, 2016

Related U.S. Application Data
(60) Provisional application No. 62/255,406, filed on Nov. 14, 2015.

(51) Int. Cl. A61K 8/41; A61K 8/42; C09D 7/12; A61Q 5/00; A61Q 19/00; C09K 3/16; A61K 8/34; A61K 8/36

(52) U.S. Cl. CPC A61K 8/416; A61K 8/345; A61K 8/42; A61K 8/36; A61K 8/34; A61Q 5/00; A61Q 19/007; C09K 3/16; C09D 7/1233; A61K 2800/20

(57) ABSTRACT

An anti-static spray comprises a base fluid, a quaternary ammonium cation, one or more moisture-retaining components, and one or more preservatives. The base fluid is water present in an amount of about 70% to about 95% by weight. The quaternary ammonium cation is cetrimonium chloride present in an amount of about 5% to about 10%. The one or more moisture-retaining components comprises glycerin in an amount of about 3% to about 7%, and panthenol in an amount of about 0.1% to about 5%. The one or more preservatives comprises sodium lactate in an amount of about 0.1% to about 3%, and phenoxyethanol in an amount of about 0.1% to about 3%.

### CROSS-REFERENCE TO RELATED APPLICATIONS

This application claims the benefit of U.S. Provisional Patent Application No. 62/255,406, filed Nov. 14, 2015, which is incorporated herein by reference in its entirety.

### TECHNICAL FIELD

The present invention relates to anti-static compounds. Specifically, the invention relates to an anti-static spray that has additional moisturizing and conditioning effects.

### BACKGROUND

The buildup of static charge in materials can present numerous issues, including static cling and an increased risk of the discharge of static electricity, both of which can be a great inconvenience to people. As a result, a variety of anti-static treatments exist, including anti-static films and sprays. However, these anti-static treatments tend to suffer from a variety of issues. For example, the anti-static affect in many of these products do not last very long, requiring a user to reapply the product in a short timeframe. Moreover, many of these treatments are harmful to the environment, animals, certain materials such as fabrics or leathers, and even humans. Some of the anti-static sprays or films may be flammable, and are very often only safe in connection with a small number of different uses. Accordingly, an improved anti-static spray is needed that addresses these issues.

### SUMMARY

According to aspects of the present invention, an anti-static spray comprises about 70% to about 95% by weight of a base fluid, about 5% to about 10% by weight of a quaternary ammonium cation, one or more moisture-retaining components, and one or more preservatives.

According to further aspects of the present invention, the quaternary ammonium cation is cetrimonium chloride in an amount of about 7% by weight.

According to further aspects of the present invention, the one or more moisture-retaining components comprise glycerin in an amount of about 5% by weight and panthenol in an amount of about 0.5% by weight.

According to further aspects of the present invention, the one or more preservatives comprises sodium lactate in an amount of about 1% by weight and phenoxyethanol in an amount of about 0.99% by weight.

According to further aspects of the present invention, an anti-static spray comprises about 70% to about 95% by weight of water, about 5% to about 10% by weight of cetrimonium chloride, about 3% to about 7% by weight of glycerin, about 0.1% to about 5% by weight of panthenol, about 0.1% to about 3% by weight of sodium lactate, and about 0.1% to about 3% by weight of phenoxyethanol.

These and other capabilities of the invention, along with the invention itself, will be more fully understood after a review of the following figures, detailed description, and claims.

### DETAILED DESCRIPTION

While this invention is susceptible of embodiment in many different forms, described in detail herein are preferred embodiments of the invention with the understanding that the present disclosure is to be considered as an exemplification of the principles of the invention and is not intended to limit the broad aspect of the invention to the embodiments illustrated. For purposes of the present detailed description, the singular includes the plural and vice versa (unless specifically disclaimed); the words “and” and “or” shall be both conjunctive and disjunctive; the word “all” means “any and all”; the word “any” means “any and all”; and the word “including” means “including without limitation.” Additionally, the singular terms “a,” “an,” and “the” include plural referents unless context clearly indicates otherwise.

According to aspects of the present disclosure, an anti-static spray comprises a base fluid, an anti-static agent, and one or more of a variety of supplementary compounds, including moisture-retaining components such as moisturizers and humectants, as well as preservatives and germicides. The anti-static spray according to aspects of the present disclosure is generally free from all formaldehyde donors, sulfates, butane propellants, glycol, petroleum, derived phthalates, and gluten. The anti-static spray is also non-flammable, non-toxic, and is made from natural ingredients.

According to aspects of the present disclosure, the base fluid of the anti-static spray is present in an amount of from about 70% to about 95% by weight. In some embodiments, the base fluid is present in an amount of about 85% by weight. In some embodiments, the base fluid is present in an amount of 85.51% by weight. According to aspects of the present disclosure, the base fluid is distilled or deionized water.

According to aspects of the present disclosure, the anti-static agent is present in the anti-static spray in an amount of about 5% to about 10% by weight. In some embodiments, the anti-static agent is present in an amount of about 7% by weight. It is contemplated that the anti-static agent is a quaternary ammonium cation, such as cetrimonium chloride or behentrimonium chloride. These anti-static agents generally consist of a head group and multiple alkyl or aryl chains, with a positive charge being located at the head group. This positive charge attracts negative charges that can build up in hair or other materials, which helps to prevent the accumulation of static in the materials and prevents undesired effects such as static cling and static discharge. Quaternary ammonium cations such as behentrimonium chloride or cetrimonium chloride are non-toxic and safe for use with a variety of materials, including humans, fabrics, and leathers.

The anti-static spray according to aspects of the present disclosure also includes a variety of supplementary compounds that provide additional benefits. For example, the anti-static spray may include one or more moisture-retaining components such as humectants and moisturizers. Humectants are hygroscopic materials that are generally used to increase the moisture content of a material. Hygroscopic materials are materials that are capable of absorbing moisture from the air and generally include one or more hydrophilic groups. Humectants generally form hydrogen bonds with water molecules in the air and increase the amount of water that the outermost layer of the skin can hold. Thus, the moisture-retaining components can provide additional benefits, such as moisturizing or conditioning effects. However, the moisture-retaining components can also improve the anti-static effect of the anti-static spray by reducing the rate at which the anti-static spray evaporates. For example, when the anti-static spray is applied to a person's hair or skin, the moisture-retaining components help keep the anti-static spray on the person's hair or skin so that the anti-static effect can last longer.

According to aspects of the present disclosure, the anti-static spray comprises one or more of a variety of supplementary compounds such as moisture-retaining components. It is contemplated that one moisture-retaining component in the anti-static spray is glycerin. In one embodiment, glycerin is present in the anti-static spray in an amount of about 3% to about 7% by weight. In another embodiment, glycerin is present in an amount of about 5% by weight. A larger concentration or amount of glycerin (for example, greater than about 7% by weight) can cause the anti-static spray to become too slippery. When the anti-static spray is too slippery, the spray has a tendency to slide or run off of the surface it has been applied to, thereby lessening the magnitude of the anti-static effect. Conversely, a smaller concentration of glycerin (for example, less than about 3% by weight) can leave the anti-static spray too tacky to be applied effectively. The glycerin in the anti-static spray imparts a smoothing effect to the anti-static spray and can slow or prevent excessive drying and evaporation of the anti-static spray, thus increasing the duration of the anti-static effect.

According to further aspects of the present disclosure, the moisture-retaining component in the anti-static spray is panthenol. Panthenol is hygroscopic and is able to easily penetrate into skin where it can bind with water molecules to prevent them from evaporating, thus serving to increase the moisture content of the material it is being applied to. According to aspects of the present disclosure, it has been found that from about 0.1% to about 5% by weight of panthenol provides effective moisturizing properties to the anti-static spray. According to further aspects of the present disclosure, it has been found that about 1% by weight of panthenol provides effective moisturizing properties to the anti-static spray.

According to aspects of the present disclosure, an embodiment of the anti-static spray comprises both glycerin and panthenol to provide the benefits of both components. In one embodiment, the anti-static spray comprises glycerin in an amount of about 3% to about 7% by weight and panthenol in an amount of about 0.1% to about 5% by weight. In another embodiment, the anti-static spray comprises glycerin in an amount of about 5% by weight and panthenol in an amount of about 0.5% by weight.

Humectants and moisturizers are uncommon components of anti-static compounds. Glycerin and panthenol have been found by the inventor to provide effective and beneficial moisturizing properties to the anti-static spray. Further, these supplementary compounds do not reduce the anti-static properties of the cetrimonium chloride and help make the anti-static effect of the spray last longer by reducing any evaporation of the anti-static spray. These beneficial properties allow the anti-static spray as described herein to be effective when applied to hair and skin, as well as surfaces such as clothing. The anti-static spray can thus act as a moisturizer for a person's hair or skin, while at the same time providing an anti-static effect and reduce the amount of static buildup in the person's hair or skin.

According to aspects of the present disclosure, the anti-static spray further comprises one or more preservatives or germicides. It is contemplated that one type of preservative in the anti-static spray is sodium lactate. In one embodiment, sodium lactate is present in the anti-static spray in an amount of about 0.1% to about 3% by weight. In a further embodiment, sodium lactate is present in an amount of about 1% by weight. When sodium lactate is present in these amounts, sodium lactate helps to lengthen the shelf-life of the anti-static spray from about six months to about one year by preventing the growth of bacteria and fungi. In addition to preventing the growth of bacteria and fungi in the anti-static spray, sodium lactate may also act as an acidity regulator.

According to aspects of the present disclosure, the anti-static spray includes a second type of preservative, such as phenoxyethanol. In one embodiment, phenoxyethanol is present in the anti-static spray in an amount of about 0.1% to about 3% by weight. In another embodiment, phenoxyethanol is present in an amount of about 0.99% by weight. It has been found that while sodium lactate may be an effective preservative for about one year, after such time period the beneficial effects of the sodium lactate are diminished. The presence of phenoxyethanol in the anti-static spray helps to increase the shelf-life of the anti-static spray to about two years. Furthermore, the combination of both sodium lactate and phenoxyethanol helps to prevent the presence of a large majority of different types of bacteria, fungi, and other undesirable growths, whereas a compound containing only sodium lactate or only phenoxyethanol may only prevent a certain subset of growths.

According to aspects of the present disclosure, an embodiment of the anti-static spray comprises both sodium lactate and phenoxyethanol to provide the benefits of both components. According to aspects of the present disclosure, an embodiment of the anti-static spray comprises sodium lactate in an amount of about 0.1% to about 3% by weight and phenoxyethanol in an amount of about 0.1% to about 3% by weight. According to aspects of the present disclosure, another embodiment of the anti-static spray comprises sodium lactate in an amount of about 1% by weight and phenoxyethanol in an amount of about 0.99% by weight.

The anti-static spray may also include one or more fragrances to improve the odor. Often, anti-static sprays have a strong, unpleasant odor. The addition of fragrance can improve the odor.

Furthermore, the anti-static spray can optionally be mixed with a small amount of citric acid to balance the pH of the anti-static spray. The pH of the anti-static spray generally should be in a range of from about 5 to about 7.

Thus, according to aspects of the present disclosure, the anti-static spray comprises about 70% to about 95% of a base fluid, about 5% to about 10% by weight of a quaternary ammonium cation, one or more moisture-retaining components, and one or more preservatives. It is contemplated that the base fluid is deionized or distilled water in an amount of about 85%. It is further contemplated that the quaternary ammonium cation is cetrimonium chloride in an amount of about 7%. In one embodiment, the one or more moisture retaining components comprises (i) glycerin in an amount of about 3% to about 7%; (ii) panethenol in an amount of about 0.1% to about 5%; (iii) or any combination of (i) and (ii). In another embodiment, the one or more moisture retaining components comprises (i) glycerin in an amount of about 5% (ii) panethenol in an amount of about 0.5%; (iii) or any combination of (i) and (ii).

In a further embodiment, the one or more preservatives comprises (i) sodium lactate in an amount of about 0.1% to about 3%; (ii) phenoxyethanol in an amount of about 0.1% to about 3%; or (iii) any combination of (i) and (ii). In yet another embodiment, the one or more preservatives comprises (i) sodium lactate in an amount of about 1%; (ii) phenoxyethanol in an amount of about 0.99%; or (iii) any combination of (i) and (ii). According to further aspects of the present disclosure, the anti-static spray comprises deionized or distilled water in an amount of about 85.51%, cetrimonium chloride in an amount of about 7%, glycerin in an amount of about 5%, panthenol in an amount of about 0.5%, sodium lactate in an amount of about 1%, and phenoxyethanol in an amount of about 0.99%.

To prepare the anti-static spray, the base fluid should first be poured or otherwise placed in a container. Following this, the quaternary ammonium cation, the moisture-retaining component(s), and the preservative(s) may all be added to the container. Once the ingredients are present, the anti-static spray should be mixed thoroughly to ensure a uniform distribution of the ingredients. Using a propeller mixer for about thirty minutes is generally sufficient to ensure a uniform distribution of the ingredients. Drops of fragrance can then be added to adjust the odor of the anti-static spray as needed. Finally, the pH of the anti-static spray should be tested, and citric acid should be added to the anti-static spray as needed or desired to adjust the pH into the desired range. The anti-static spray should have a viscosity and appearance similar to that of water. In other aspects according to the present disclosure, the ingredients may be added to the container in any desired order.

The anti-static spray may be applied to a variety of surfaces to reduce static. Importantly, the anti-static spray does not need to be in propellant form to be effective. Rather, the anti-static spray can be applied by simply rubbing the composition onto a desired material or surface. For example, the anti-static spray can be applied to clothing to reduce static that may build up while sleeping or throughout the day. The anti-static spray may also be applied to hair or skin to reduce static. The anti-static spray according to aspects of the present invention can impart other beneficial effects to hair or skin as well. The moisture-retaining components in the anti-static spray can help moisturize and condition a person's hair or skin, in addition to reducing any static buildup. The spray may also be applied to clothing, fabrics, leather, plastics, wood, rubber, and metals with no negative effects, and is safe for humans and animals. For example, the anti-static spray can be applied to a car door handle to prevent a person from being shocked when they touch the handle. The anti-static spray can also be added to a load of clothing or blankets in the washing machine to reduce static buildup that is normally present once the articles are dried. It has been found that the anti-static spray is long-lasting and can continue to provide the anti-static effect for days after the initial application. Furthermore, the anti-static spray is non-flammable and has actually been found to act as a flame retardant. The anti-static spray is also environmentally and ozone friendly.

While the term anti-static “spray” is used, one of skill in the art will recognize that the disclosed anti-static composition does not need to be sprayed, and can be applied to surfaces in other ways.

While the present invention has been described with reference to one or more particular embodiments, those skilled in the art will recognize that many changes may be made thereto without departing from the spirit and scope of the present invention. Each of these embodiments and obvious variations thereof is contemplated as falling within the spirit and scope of the invention. It is also contemplated that additional embodiments according to aspects of the present invention may combine any number of features from any of the embodiments described herein.

What is claimed is:

1. An anti-static spray, comprising:
about 70% to about 95% by weight of a base fluid;
about 5% to about 10% by weight of a quaternary ammonium cation;
one or more moisture-retaining components; and
one or more preservatives.

2. The anti-static spray of claim 1, wherein the quaternary ammonium cation is cetrimonium chloride.

3. The anti-static spray of claim 2, where the cetrimonium chloride is present in an amount of about 7% by weight.

4. The anti-static spray of claim 1, wherein the one or more moisture-retaining components comprise glycerin present in an amount of about 3% to about 7% by weight.

5. The anti-static spray of claim 4, wherein the glycerin is present in an amount of about 5% by weight.

6. The anti-static spray of claim 1, wherein the one or more moisture-retaining components comprise panthenol present in an amount of about 0.1% to about 5% by weight.

7. The anti-static spray of claim 6, wherein the panthenol is present in an amount of about 0.5% by weight.

8. The anti-static spray of claim 1, wherein the one or more moisture-retaining components comprise glycerin present in an amount of about 3% to about 7% by weight and panthenol present in an amount of about 0.1% to about 5% by weight.

9. The anti-static spray of claim 1, wherein the one or more moisture-retaining components comprise glycerin present in an amount of about 5% by weight and panthenol present in an amount of about 0.5% by weight.

10. The anti-static spray of claim 1, wherein the one or more preservatives comprise sodium lactate present in an amount of about 0.1% to about 3% by weight.

11. The anti-static spray of claim 10, wherein the sodium lactate is present in an amount of about 1% by weight.

12. The anti-static spray of claim 1, wherein the one or more preservatives comprise phenoxyethanol present in an amount of about 0.1% to about 3% by weight.

13. The anti-static spray of claim 12, wherein the phenoxyethanol is present in an amount of about 0.99% by weight.

14. The anti-static spray of claim 1, wherein the one or more preservatives comprise sodium lactate present in an amount of about 0.1% to about 3% by weight and phenoxyethanol present in an amount of about 0.1% to about 3% by weight.

15. The anti-static spray of claim 14, wherein the one or more preservatives comprise sodium lactate present in an amount of about 1% by weight and phenoxyethanol present in an amount of about 0.99% by weight.

16. An anti-static spray, comprising:
about 70% to about 95% by weight of water;
about 5% to about 10% by weight of cetrimonium chloride;
about 3% to about 7% by weight of glycerin;
about 0.1% to about 5% by weight of panthenol;
about 0.1% to about 3% by weight of sodium lactate; and
about 0.1% to about 3% by weight of phenoxyethanol.
